(19) United States
(12) Patent Application Publication
(10) Pub. No.: US 2017/0042478 A1
(43) Pub. Date: Feb. 16, 2017

(54) DEVICE AND METHOD OF TRANSMITTING AND DETECTING ELECTROMAGNETIC SIGNAL PROPAGATING THROUGH CRANIAL SUTURES

(71) Applicant: Chong Qing Born-Fuke Medical Equipment Co., Ltd, New North Zone, Chongqing (CN)

(72) Inventors: Yi Zheng, Cold Spring, MN (US); Hui Jiang, Chongqing (CN); Qi Wu, Chongqing (CN); Eugene Edward Zheng, Cold Spring, MN (US); Jiahua Peng, Chongqing (CN); Jiansong Zhou, Chongqing (CN)

(21) Appl. No.: 15/111,163

(22) PCT Filed: Jul. 15, 2015
(86) PCT No.: PCT/CN15/84135

§ 371 Date: Jul. 12, 2016

(30) Foreign Application Priority Data
Apr. 15, 2015 (CN) 201510178437.9

(51) Int. Cl. A61B 5/00; A61B 5/04

(52) U.S. Cl. CPC A61B 5/6803; A61B 5/04001; A61B 5/04008

(57) ABSTRACT

The embodiments of present invention disclose a device and a method of transmitting and detecting electromagnetic signal propagating through cranial sutures. The device comprises: a positioning strap and a differential amplifier circuit, wherein, the positioning strap on the inside thereof is provided with several electrode pads; at least one of the several electrode pads functions as an emitter electrode pad, at least two of the several electrode pads function as two receiver electrode pads, which are connected to two input terminals of the differential amplifier circuit, respectively; all the electrode pads are arranged on the inside surface of the positioning strap, and the shape of distribution of the electrode pads on the positioning strap matches the shape of at least one cranial suture on a cranium of an object under examination when the strap is placed around. When the device is used for monitoring and detecting a pathological condition inside the cranium, signal integrity of received electromagnetic wave signals is greatly enhanced if the location of the electrode pads placed on the inside of the positioning strap matches the location of a cranial suture of the cranium, and the received signals are further processed by a differential amplifier circuit so that interfering noise loaded to the electrode pads is eliminated, thereby the pathological conditions inside the cranium can be reflected accurately in the measurements.

### CROSS-REFERENCE TO RELATED APPLICATION

The present application claims priority to Chinese Patent Application No. 201510178437.9 filed with the State Intellectual Property Office on Apr. 15, 2015, titled “DEVICE AND METHOD FOR SIGNAL DETECTION”, which is incorporated herein by reference in its entirety.

### FIELD OF THE INVENTION

The present invention relates to a field of signal detection technologies, and in particular, to a device and a method of transmitting and detecting electromagnetic signal propagating through cranial sutures.

### BACKGROUND OF THE INVENTION

An electromagnetic wave signal can propagate its energy in the form of a wave in a medium, and electromagnetic wave signals with the same frequency have different waveforms when propagating in different media. Therefore, electromagnetic wave signals may be utilized to differentiate between different media. Usually, a method for differentiating different media by an electromagnetic wave signal mainly comprises the steps of: first of all, emitting electromagnetic wave signals with the same frequency to each type of media under examination so that the electromagnetic wave signals propagate in the media; then, receiving the electromagnetic wave signals after they pass through the media; and finally, comparing the various received electromagnetic wave signals so as to thereby differentiate different types of media.

According to the above method, electromagnetic waves may be utilized to detect the impurity in a substance, for example, to detect edema or blood clot in a biological tissue. First of all, the waveform data of an electromagnetic wave signal with a certain frequency is obtained after propagating in an impurity-free biological tissue, and the waveform data are taken as standard data. A plurality of electrode pads adhere to the outside of the biological tissue, wherein, each of the electrode pads has a specific attribute, comprising an emitter electrode pad that emits outward an electromagnetic wave signal with a certain frequency, a receiver electrode pad that receives an electromagnetic wave signal, and a ground electrode pad. When edema or blood clot exists at a certain place inside the biological tissue, the waveform data of the electromagnetic wave signals received by the receiver electrode pad are different from the standard data, and thus it may be concluded that edema or blood clot exists on the propagation path of the electromagnetic wave signal in the biological tissue.

When an electromagnetic wave signal is utilized to measure a biological tissue, a medical care personnel needs to apply an electrode pad to a prescribed location on an object to be measured. Because different medical care persons have different levels of skills for adhering an electrode pad, the adhesions are not consistent. For example, the electrode pad is adhered to the different locations when being adhered, or the electrode pad is not adhered firmly enough so it is loose or comes off, which all may cause the interferences to the received electromagnetic wave signals, thereby the pathological state inside the biological tissue cannot be reflected accurately and truly by the measurements.

### SUMMARY OF THE INVENTION

The embodiments of the invention disclose a device and a method for signal detection.

In order to solve the technical problems of the effective transmission and receiving electromagnetic waves and the consistent adherences of electrodes, the embodiments of the invention provide the technical solutions below:

A device for signal detection, comprising: a positioning strap and a differential amplifier circuit, wherein, several electrode pads are attached on the inside of the positioning strap; at least one of the several electrode pads functions as an emitter electrode pad, at least two of the several electrode pads function as two receiver electrode pads, which are connected to two input terminals of the differential amplifier circuit, respectively; all the electrode pads are arranged on the inside surface of the positioning strap, and the placements of the electrode pads on the positioning strap match the shape of at least one cranial suture on a cranium of an object to be measured.

Preferably, the electrode pad is placed d on the positioning strap in a manner of adhesion or stitching.

Preferably, there exists one positioning strap, and the positioning strap is formed in an annular shape that is closed and can be worn around the cranium of the object to be measured.

Preferably, there exist a plurality of positioning straps, and the plurality of positioning straps are connected with one another to form a space in which the cranium of the object to be measured can be accommodated.

Preferably, the shapes of distribution of electrode pads on every positioning strap match the shapes of different cranial sutures on the cranium of the object under examination respectively.

Preferably, the positioning strap is an elastic strap.

Preferably, the differential amplifier circuit comprises a differential amplifier, for performing differential amplification on the electromagnetic wave signals received from the two receiver electrode pads to obtain a target signal.

A method for signal detection, which is applied to the above device for signal detection, comprising:

controlling an electromagnetic wave signal emitting device to emit an electromagnetic wave to an emitter electrode;

obtaining the electromagnetic wave signals received from at least two receiver electrode pads; and

performing differential amplification on the electromagnetic wave signals received from the at least two receiver electrode pads to obtain a target signal.

Based on the above technical solutions that, the device for signal detection according to the embodiments of the invention has the following beneficial effects:

When the above device for signal detection is used for detecting a situation of a brain, if the location of the electrode pads on the inside of the positioning strap coincides with one cranial suture of human brain, the electromagnetic wave signals received by the receiver electrode pads are clear, and then are further processed by the differential amplifier circuit so that interfering signals loaded to the receiver electrode pads is eliminated, thereby the error between the target signal obtained and the actual signal reflecting the pathological situation is reduced, the accuracy of the measurement result is improved, and hence the situation of the brain is accurately reflected in the measurement.

Because the shape of distribution of the electrode pads on the positioning strap matches the shape of at least one cranial suture on the cranium of the object to be measured and the blocking effect of a cranial suture on an electromagnetic wave is less than that of a cranium on the electromagnetic wave, the electrode pad on the inside of the positioning strap can emit or receive a stronger electromagnetic wave signal no matter it functions as an emitter electrode or a receiver electrode.

Additionally, the differential amplifier circuit plays a role of inhibiting interference, thus after an electromagnetic wave signal received passes through the differential amplifier circuit, external electromagnetic interferences and the interfering signals loaded to the receiving end due to the loosing or coming off of an adhered electrode pads can be eliminated, thereby the electromagnetic wave signal output from the differential amplifier circuit will be cleaner.

### BRIEF DESCRIPTION OF THE DRAWINGS

To illustrate the technical solutions of the embodiments of this invention, the drawings for the description of the embodiments are briefly introduced below. It is apparent to one of ordinary skills in the art that other drawings may also be obtained according to these drawings without creative work.

FIG. 1 is a structural diagram of a device for signal transmission and detection according to one embodiment of the invention;

FIG. 2 is a structural diagram of another device for signal transmission and detection according to one embodiment of the invention;

FIG. 3 is a schematic diagram showing that the shape of distribution of electrode pads inside a positioning strap of a device for signal detection according to one embodiment of the invention matches the shape of any cranial suture on the cranium;

FIG. 4 is a schematic diagram showing that the shape of distribution of electrode pads inside a positioning strap of a device for signal detection according to one embodiment of the invention does not match the shape of any cranial suture on the cranium;

FIG. 5 is a flow chart of a method for signal transmission and detection according to one embodiment of the invention; and

FIG. 6 is a circuit diagram of a differential amplifier circuit of a device for signal transmission and detection according to one embodiment of the invention.

### DETAILED DESCRIPTION OF THE EMBODIMENTS

In order to enable one skilled in the art better to understand the technical solutions of the embodiments of the invention and to make the above objects, the characteristics, and the advantages of the embodiments of the invention more apparent, the technical solutions in the embodiments of the invention are further described in detail below in conjunction with the drawings.

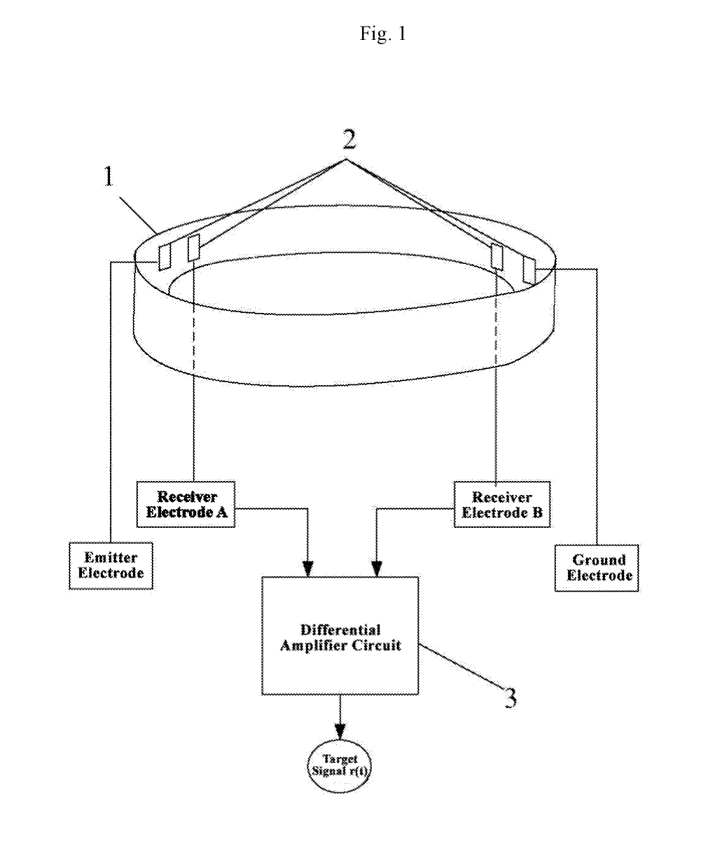

One embodiment of the invention provides a device for signal detection, which may be used especially for monitoring a brain of an object under examination. As shown in FIG. 1, the device for signal detection comprises: a positioning strap 1 and a differential amplifier circuit 3, wherein, several electrode pads 2 are placed on the inside of the positioning strap 1; at least one of the several electrode pads functions as an emitter electrode pad, which is connected to an emitter electrode.

In the device, at least two of the several electrode pads 2 function as two receiver electrode pads, which are respectively provided as receiver electrode A and receiver electrode B which are respectively connected to two input terminals of the differential amplifier circuit 3.

All electrode pads 2 on the device are placed on the inside surface of the positioning strap 1, and a shape of distribution of electrode pads 2 on the positioning strap matches a shape of at least one cranial suture on the cranium of the object under examination.

In the embodiments of the disclosure, the shape of distribution of the electrode pads refers to a shape of a connecting line for the center points of a plurality of electrode pads.

Cerebral cranium is consisted of 8 pieces of bones, i.e., a pair of parietal bones, a pair of temporal bones, a frontal bone, an ethmoid bone, a sphenoid bone and an occipital bone. The 8 pieces of bones of cerebral cranium are connected via cranial sutures to jointly form a cranial cavity. A cranial suture refers to a seam between cranium bones. The bones of an adult calvarium are jointed via saw-shaped cranial sutures, and the locations of the cranial suture are relatively stable. Collagen fiber is the main force-bearing unit of the cranial suture. Where coronal sutures exist at the connections between the frontal bone and the parietal bones on the two sides, a sagittal suture exists at the connection between the parietal bones on the two sides, lambdoid sutures exist at the connections between the parietal bones on the two sides and the occipital bone, and a scaly suture exists at the connection between the temporal bone, the parietal bone and the sphenoid bone.

The above matching of the shape of distribution of the electrode pads on the positioning strap with the shape of at least one cranial suture on the cranium of an object under examination refers to that, the shape of the connecting line for the center points of a plurality of electrode pads coincides with fully or partially the shape of at least one cranial suture on the cranium of the object under examination, or the distance between the connecting line for the center points of the electrode pads and the at least one cranial suture on the cranium is less than the width from the connecting line for the center points of the electrode pads to one side thereof, or the shape of the at least one cranial suture on the cranium of the object under examination is contained within a region of a shape formed by the plurality of electrode pads on the positioning strap, or a half or more than a half of the shape of the cranial suture is contained within the region of the shape formed by the plurality of electrode pads.

As shown in FIG. 1, in one embodiment of the invention, one positioning strap of the device is shown as the first positioning strap 1, and when the device has only one positioning strap, the positioning strap may be formed in an annular shape that is closed and may be worn around the cranium of the object under examination. As shown in FIG. 1, the first positioning strap 1 is in the form of a circular shape, thus when the detection device is used for monitoring a human brain, the first positioning strap 1 may be worn around a human cranium.

As shown in FIG. 1, the first positioning strap 1 is provided with a plurality of electrode pads 2, wherein the plurality of electrode pads 2 are divided into two parts, and as shown in FIG. 1, the two electrode pads 2 on the left may be regarded as one group, the two electrode pads 2 on the right may be regarded as another group, and the shape of distribution of each group of electrode pads may match the shape of one cranial suture of the object under examination.

In the embodiment as shown in FIG. 1, the device requires at least four electrodes for measuring the object under examination, and the four electrodes are connected to the electrode pads respectively, wherein the four electrodes are respectively:

an emitter electrode pad, which is connected to the emitter electrode, for emitting an electromagnetic wave signal to the object under examination;

two receiver electrode pads, i.e., a receiver electrode A and a receiver electrode B, for receiving the electromagnetic wave signal that passes through the object under examination, wherein the two receiver electrode pads are connected to two input terminals of a differential amplifier circuit via the receiver electrode A and the receiver electrode B; and

a ground electrode pad, i.e., a reference ground electrode.

The locations where the above four electrode pads are adhered are as shown in FIG. 1, wherein, the emitter electrode and the receiver electrode A are regarded as one group, the ground electrode and the receiver electrode B are regarded as another group and the two groups are distributed symmetrically on the inside of the positioning strap 1. Preferably, in other embodiments, the device for signal detection may be provided with a plurality of receiver electrode pads, and the locations of the plurality of receiver electrode pads are not limited to the locations of the receiver electrode A and the receiver electrode B.

As shown in FIG. 1, a differential process is performed by the differential amplifier circuit 3 on the electromagnetic wave signals received by the receiver electrode A and the receiver electrode B to obtain a target signal r(t). The above differential amplifier circuit comprises a differential amplifier, which performs differential amplification on the received electromagnetic wave signals propagating through cranial sutures.

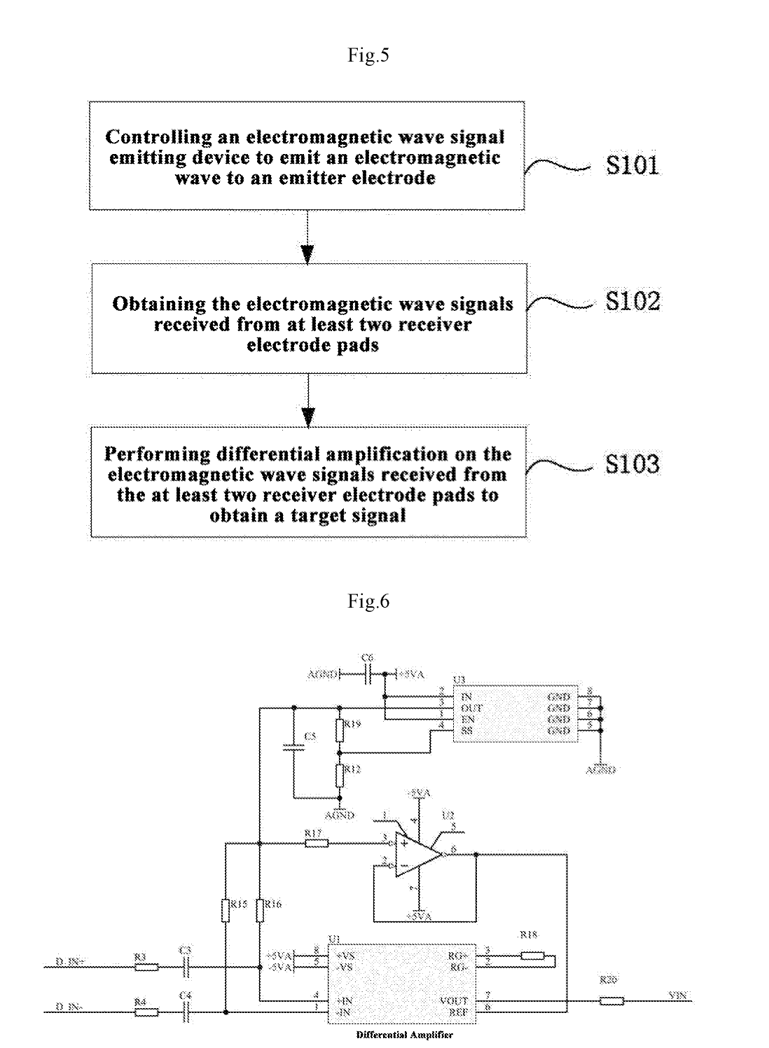

As shown in FIG. 6, it is a circuit diagram of a differential amplifier circuit according to this embodiment, which comprises a differential amplifier U1 and a common-mode output circuit consisted of U2 and U3, wherein the receiver electrode A is connected to input terminal D_IN+ of the differential amplifier circuit and functions as input terminal +IN of the differential amplifier U1 after passing through a resistor R3 and a capacitor C3, the receiver electrode B is connected to a further input terminal D_IN− of the differential amplifier circuit and functions as a further input terminal −IN of the differential amplifier U1 after passing through resistor R4 and capacitor C4, output signal VOUT is obtained after the received electromagnetic wave signal passes through differential amplifier U1, and then target signal r(t) is obtained after output signal VOUT passes through resistor R20.

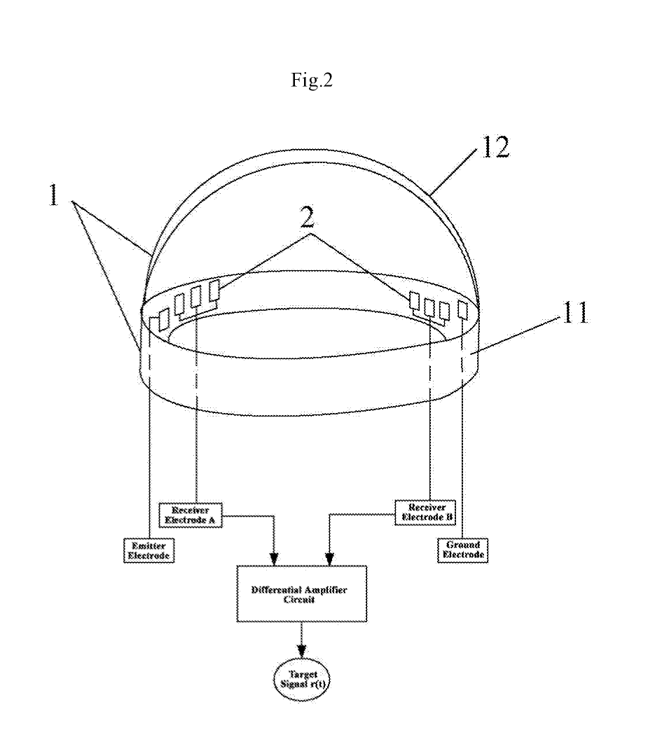

As shown in FIG. 2, in a further embodiment of the present application, the transmission and detection device may comprise: the first positioning strap 11 and the second positioning strap 12, wherein, for the detailed description of the first positioning strap 11, reference may be made to the description in the previous paragraphs; the second positioning strap 12 is in a half annular shape, both ends of the second positioning strap 12 are fixed on the first positioning strap 11, and the fixing positions for the two ends of the second positioning strap 12 lie on the opposite two sides of the first positioning strap 11, respectively.

In one embodiment of the present application, the second positioning strap 12 may be provided with electrode pads or without electrode pad, which may be selected freely depending on actual demands. When the second positioning strap 12 is provided with electrode pads, a shape of distribution of electrode pads on the second positioning strap 12 is different from the shape of distribution of electrode pads on the first positioning strap 11, that is, the shapes of distributions of electrode pads on every positioning strap match the shapes of different cranial sutures on the cranium of the object under examination, respectively.

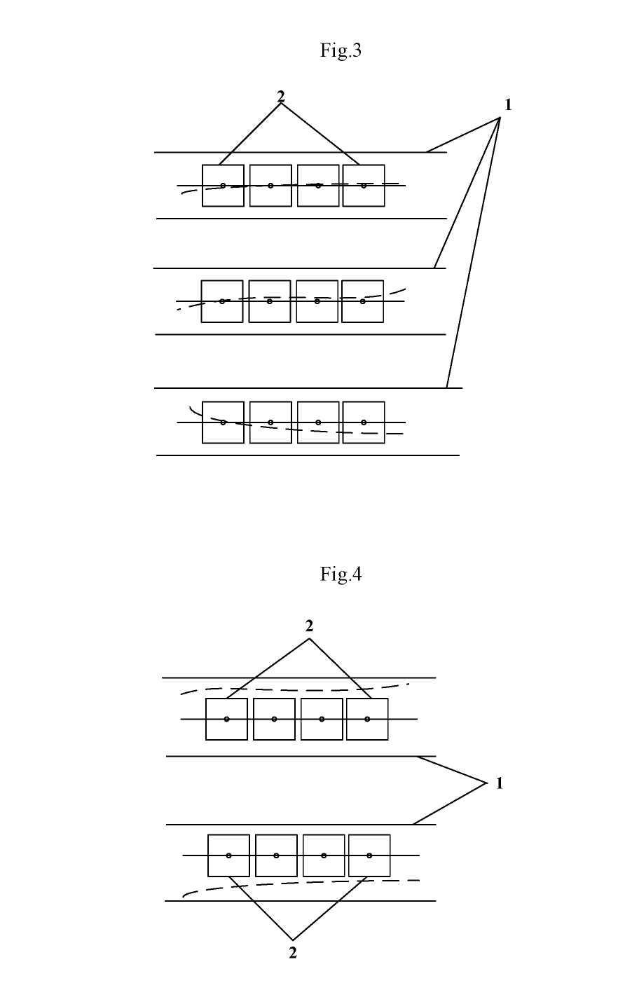

As shown in FIG. 3, three cases are illustrated in which the shape of distribution of the electrode pads on the positioning strap matches the shape of at least one cranial suture on the cranium of the object under examination, wherein, the solid line represents the connecting line for the center points of four electrode pads, and the dashed line represents a cranial suture on the cranium. FIG. 3 only shows three cases in which the shape of the center line of the electrode pads matches the shape of one cranial suture, and other cases like or similar to FIG. 3 also pertain to the scope that the shape of distribution of the electrode pads on the positioning strap matches the shape of at least one cranial suture on the cranium of the object under examined.

FIG. 4 illustrates two cases in which the shape of distribution of the electrode pads on the positioning strap does not match the shape of at least one cranial suture on the cranium of an object under examination, wherein, the solid line represents the connecting line for the center points of four electrode pads, and the dashed line represents one cranial suture on the cranium. FIG. 4 only illustrates two cases in which the shape of the center line of the electrode pads does not match the shape of one cranial suture, and other cases like or similar to FIG. 4, for example, a case, in which the shape of any cranial suture on the cranium does not pass through the region of the shape formed by a plurality of electrode pads, also pertains to the scope that the shape of distribution of the electrode pads on the positioning strap does not match the shape of at least one cranial suture on the cranium of an object under examination.

In other embodiments of this disclosure, the device for signal detection may also comprise a plurality of positioning straps according to actual demand, whereas the plurality of positioning straps are connected to form a space in which the cranium of the object under examination can be accommodated.

Additionally, in order to improve the comfortability during wearing, all the above positioning straps may be elastic, so that the electrode pads on the inside of the positioning strap may closely contact the skin of an object under examination when being in use.

In the embodiments of this disclosure, the electrode pad 2 may be fixed to the positioning strap in the manner of adhesion, or the electrode pad 2 may be stitched to the positioning strap in the manner of stitching, and the like.

In the use of the device for signal transmission and detection according to the embodiments of the invention, the positioning strap may be placed to the cranium of an object under examination, so that the several electrode pads on the positioning strap may be attached to the cranial suture, and the locations of the two groups of electrode pads on the positioning strap 11 may fit the human scaly sutures, respectively.

When a certain electrode pad on the positioning strap is used as an emitter electrode, an electromagnetic wave signal may be emitted into the cranium via the cranial suture; and when a certain electrode pad on the positioning strap is used as a receiver electrode, the electromagnetic wave signal transmitted from inside the cranium may be received via the cranial suture.

Because the main force-bearing unit of the cranial suture is collagen fiber, in comparison with the osseous structure of the cranium, the blocking effect of a cranial suture on an electromagnetic wave is less than the blocking effect of the cranium on the electromagnetic wave. Therefore, when a certain electrode pad on the positioning strap is used as an emitter electrode, the electromagnetic wave signal entering the cranial cavity after being emitted has a higher intensity; and when a certain electrode pad on the positioning strap is used as a receiver electrode, the received electromagnetic wave signal propagated through the cranial cavity has a higher intensity. Thus, when the device is used for monitoring cerebral edema or hydrocephalus, the detected electromagnetic wave signal is stronger, thereby the monitoring results based on the detected electromagnetic wave signals is more accurate.

Additionally, when the adhesion of the electrode pad of the emitter electrode or the ground electrode looses, some interfering signals, including interfering noises, may be generated from the receiver electrode A and the receiver electrode B. The interfering noises generally are loaded with equal values at the same time to the two receiver electrodes. When passing through the differential amplifier circuit 3, the interfering signals from the receiver electrode A and the receiver electrode B will be inhibited due to a very strong capability of inhibition by the differential amplifier circuit 3 on common-mode signals, that is, the outputted target signal r(t) is the difference value between the electromagnetic waves received by the receiver electrode A and the receiver electrode B. Because the interfering noises loaded to the above two signals have equal values, the difference value due to the interfering noise is zero. Thus, the interfering noises are inhibited after passing through the differential amplifier circuit 3, and a clean target signal is obtained, thereby the influence of the interfering noise on the measurement result due to the loose of electrode pad adhesion is eliminated, the accuracy of the measurement result is improved, and the status of cerebral edema is more accurately reflected in the measurement results.

Based on the above embodiments for the device, one embodiment of the disclosure further provides a method for signal transmission and detection through cranial sutures. As shown in FIG. 5, the method comprises the steps of:

Step S101: controlling an electromagnetic wave signal emitting device to emit an electromagnetic wave to an emitter electrode;

The electromagnetic wave signal emitting device emits a prescribed electromagnetic wave signal via an emitter electrode to an object under examination, wherein, the above electromagnetic wave signal emitting device may be a signal generator, which may select electromagnetic wave signals with a specific frequency. Before emitting an electromagnetic wave signal, an emitter electrode pad on the positioning strap communicates with the electromagnetic wave signal emitting device by a control switch. Thereby, in this step, at least one electrode pad may communicate with the electromagnetic wave signal emitting device by a control switch.

Step S102: receiving the electromagnetic wave signals propagating through cranial sutures from at least two receiver electrode pads;

As shown in FIG. 5, when there exist a plurality of receiver electrode pads, several electrode pads that are in the same group as the emitter electrode may be connected to receiver electrode A, and several electrode pads that are in the same group as the ground electrode may be connected to receiver electrode B, wherein the receiver electrode A and the receiver electrode B functions as two input ends of a differential amplifier circuit. Before Step S102 is performed, all the receiver electrode pads may communicate with the receiver electrodes by a control switch, thereby, in this step, at least two receiver electrode pads on the positioning strap may communicate with an electromagnetic wave signal receiving device by a control switch.

Step S103: performing differential amplification on the electromagnetic wave signals received from the at least two receiver electrode pads to obtain a target signal r(t);

Differential amplification is performed on the electromagnetic wave signals received from at least two receiver electrode pads, thereby interfering signals due to adhesion error or loose of electrode pads is reduced. Because the differential amplifier circuit has a function of inhibiting electromagnetic interference, the electromagnetic wave signals is clearer after being processed by the differential amplifier circuit, the interfering signals being removed so that the received signals may be cleaner. Then, pathological conditions inside the cranium of the object under examination can be assessed using digitized measurements for analyzation and computation such as orthogonalization process, etc.

Because the shape of distribution of the electrode pads on the positioning strap matches the shape of one cranial suture on the cranium of the object under examination, when the detection device is worn on the cranium of the object under examination, by adjusting the location of the detection device, the location of the emitter electrode pads may be rendered to correspond to the location of one cranial suture on the cranium of the object under examination, that is, the location of the connecting line for the center points of a plurality of electrode pads may be rendered to lie on a cranial suture, and thereby the electrode pads may be attached to the skin of the object under examination along the cranial suture.

Because the main force-bearing unit of the cranial suture is collagen fiber, in comparison with the osseous structure of the cranium, the blocking effect of a cranial suture on an electromagnetic wave is less than the blocking effect of the cranium on the electromagnetic wave. Therefore, when the emitter electrode pad is used as an emitter electrode, the electromagnetic wave signal propagating through cranial sutures and entering the cranial cavity after being emitted has a higher intensity.

Similarly, when the receiver electrode pad is used as a receiver electrode, the received electromagnetic wave signal propagating through the cranial cavity and cranial sutures has a higher intensity.

Therefore, when the method is used for monitoring cerebral edema or hydrocephalus, the detected electromagnetic wave signal is stronger, thereby the result measured according to the electromagnetic wave signal is more accurate and robust, and the status of intracranial conditions can be reflected more accurately by the measurements.

The above descriptions only show some specific embodiments of the invention. It should be noted that, for one of ordinary skills in the art, various improvements and modifications may also be made without departing from the principles of the invention, and all these improvements and modifications should be regarded as falling into the protection scope of the invention.

1-8. (canceled)

9. A device for signal transmission and detection, comprising: a positioning strap and a differential amplifier circuit, wherein:
a plurality of electrode pads are placed on the inside of the positioning strap;
a signal generator to generate an electromagnetic signal with a prescribed amplitude and frequency;
at least one of the plurality of electrode pads functions as an emitter electrode pad that is connected with the signal generator;
at least two of the plurality of electrode pads function as two receiver electrode pads, which are connected to two input terminals of the differential amplifier circuit, respectively;
at least one electrode pad functions as a ground electrode pad;
all the electrode pads are arranged on the inside surface of the positioning strap, and a shape of distribution of the electrode pads on the positioning strap matches a shape of at least one cranial suture on a cranium of an object under examination.

10. The device according to claim 9, wherein the electrode pads are fixed on the positioning strap in a manner of adhesion

11. The device according to claim 9, wherein the electrode pads are fixed on the positioning strap in a manner of stitching.

12. The device according to claim 9, wherein when there exists one positioning strap;
the positioning strap is formed in an annular shape that is closed and can be worn around the cranium of the object under examination.

13. The device according to claim 9, wherein when there exists a plurality of positioning straps;
the plurality of positioning straps are connected with one another to form a space in which the cranium of the object under examination is able to be accommodated.

14. The device according to claim 13, wherein the shapes of distributions of electrode pads on every positioning strap match the shapes of different cranial sutures on the cranium of the object under examination, respectively.

15. The device according claim 9, wherein, the positioning strap is an elastic strap.

16. The device according to claim 9, wherein:
the differential amplifier circuit comprises a differential amplifier, for performing differential amplification on the electromagnetic wave signals received from the two receiver electrode pads to obtain a target signal.

17. A method for signal transmission and detection, which is applied to the device for signal transmission and detection according to claim 9, the method comprising:
placing electrode pads on the inside surface of the positioning strap;
placing the positioning strap around a head so that a shape of the distribution of the electrodes pads on the positioning strap matches a shape of at least one cranial suture on a cranium of an object under examination;
selecting one of the electrode pads as a transmitting electrode pad by using a control switch;
selecting two of the electrode pads as receiver electrode pads by using the control switch;
selecting one of the electrode pads as a ground electrode pad by using the control switch;
controlling an electromagnetic wave signal emitting device to emit an electromagnetic wave to the emitter electrode pad;
receiving the electromagnetic wave signals from at least two receiver electrode pads; and
performing differential amplification on the electromagnetic wave signals received from the at least two receiver electrode pads to obtain a target signal.

18. The method according to claim 17, wherein the electrode pads are fixed on the positioning strap in a manner of adhesion

19. The method according to claim 17, wherein the electrode pads are fixed on the positioning strap in a manner of stitching.

20. The method according to claim 17, wherein when there exists one positioning strap;
the positioning strap is formed in an annular shape that is closed and can be worn around the cranium of the object under examination.

21. The method according to claim 17, wherein when there exists a plurality of positioning straps;
the plurality of positioning straps are connected with one another to form a space in which the cranium of the object under examination is able to be accommodated.

22. The method according to claim 21, wherein the shapes of distributions of electrode pads on every positioning strap match the shapes of different cranial sutures on the cranium of the object under examination, respectively.

23. The method according to claim 17, wherein, the positioning strap is an elastic strap.

24. The method according to claim 17, wherein:
the differential amplifier circuit comprises a differential amplifier, for performing differential amplification on the electromagnetic wave signals received from the two receiver electrode pads to obtain a target signal.
